SEQUENCE LISTING (11 sequences)

SEQ ID NO: 1 - protein, 4 aa, Artificial
  N-terminal part of SplB protease recognition site
WELQ

SEQ ID NO: 2 - protein, 6 aa, Artificial
  N-terminal part of HRV3C protease recognition site
LEVLFQ

SEQ ID NO: 3 - protein, 6 aa, Artificial
  N-terminal part of TEV protease recognition site
ENLYFQ

SEQ ID NO: 4 - protein, 6 aa, Artificial
  N-terminal part of TVMV protease recognition site
ETVRFQ

SEQ ID NO: 5 - protein, 83 aa, Artificial
  Split domain of the FbaB-type Fibronectin- binding protein of Streptococcus pyogenes
DSATHIKFSKRDEDGKELAGATMELRDSSGKTISTWISDGQVKDFYLYPGKYTFVETAAPDGYEVATAITFTVNEQGQVTVNG

SEQ ID NO: 6 - protein, 12 aa, Artificial
  Split domain of the FbaB-type Fibronectin-like binding protein of Streptococcus pyogenes
AHIVMVDAYKPT

SEQ ID NO: 7 - protein, 195 aa, Artificial
  Affinity clamp protein
GSPELGFSISGGVGGRGNPFRPDDDGIFVTRVQPEGPASKLLQPGDKIIQANGYSFINIEHGQAVSLLKTFQNTVELIIVREVGNGAKQEIRVRVEKDGGSGGVSSVPTNLEVVAATPTSLLISWDAYRELPVSYYRITYGETGGNSPVQEFTVPGSKSTATISGLKPGVDYTITVYAHYNYHYYSSPISINYRT

SEQ ID NO: 8 - protein, 8 aa, Artificial
  ARVCF peptide
PQPVDSWV

SEQ ID NO: 9 - protein, 8 aa, Artificial
  ARVCF peptide
RGSIDSWV

SEQ ID NO: 10 - protein, 5 aa, Artificial Sequence
  ARVCF peptide
VDSWV

SEQ ID NO: 11 - protein, 4 aa, Artificial Sequence
  ARVCF peptide
DSWV

(19) United States
(12) Patent Application Publication
(10) Pub. No.: US 2018/0141972 A1
(43) Pub. Date: May 24, 2018

(54) NATIVE PROTEIN PURIFICATION TECHNOLOGY

(71) Applicant: AGENCY FOR SCIENCE, TECHNOLOGY AND RESEARCH, Singapore (SG)

(72) Inventors: Saurabh Rajendra NIRANTAR, Singapore (SG); Farid John GHADESSY, Singapore (SG)

(21) Appl. No.: 15/574,481

(22) PCT Filed: May 13, 2016
(86) PCT No.: PCT/SG2016/050226

§ 371 Date: Nov. 15, 2017

(30) Foreign Application Priority Data
May 15, 2015 (SG) 10201503873T

(51) Int. Cl. C07K 1/22; C12N 15/85

(52) U.S. Cl. CPC C07K 1/22; C12N 15/85; C12N 2015/8518; C07K 2319/50; C07K 2319/70; C07K 2319/21

(57) ABSTRACT

The present invention relates to an isolated polypeptide comprising (a) a protein of interest; (b) a first member of a pair of binding partners; (c) an affinity tag for immobilizing the polypeptide on a solid support; and (d) a modified endoprotease recognition site, wherein the modified endoprotease recognition site is located directly adjacent to the N-terminal amino acid of the protein of interest and comprises or only consists of the amino acid sequence N-terminal of the cleavage site of the native endoprotease recognition site. The present invention also relates to a nucleic acid encoding the above polypeptide and a host cell thereof, a method for isolating a protein of interest using the above polypeptide as a fusion partner and a protease fusion protein with the second member of the pair of binding partners and kits thereof. In addition, a method of degrading a target protein, a method of treatment and use of a fusion protease protein comprising a protease and a target protein binding element are also disclosed.

### FIELD OF THE INVENTION

The present invention lies in the field of biochemistry and relates to an isolated polypeptide comprising (a) a protein of interest; (b) a first member of a pair of binding partners; (c) an affinity tag for immobilizing the polypeptide on a solid support; and (d) a modified endoprotease recognition site, wherein the modified endoprotease site is located directly adjacent to the N-terminal amino acid of the protein of interest and comprises or only consists of the amino acid sequence N-terminal of the cleavage site of the native endoprotease recognition site. The present invention also relates to a nucleic acid encoding the above polypeptide, a host cell comprising the nucleic acid of the invention, a method for isolating a protein of interest using the above polypeptide as a fusion partner and to a kit comprising an expression vector and a protease fusion protein.

### BACKGROUND OF THE INVENTION

Protein purification is an essential task in academia as well as industry. This is usually achieved by fusing various affinity tags like His-tag, MBP etc. to the gene of interest, followed by protein expression and purification using a column/binding matrix which specifically binds to and retains the fused affinity tag. While this process has been effectively optimized over decades, it tends to leave behind the affinity tag fused to the protein of interest, which may interfere in downstream application or give rise to an immune response etc.

The tag may be removed by placing a protease site between the protein of interest and the affinity tag; however, most proteases require a specific amino acid sequence both before and after the site of cleavage. Thus a small peptide sequence is still retained after protease cleavage. For example, PreScission Protease (HRV3C protease) requires the sequence LEVLFQ| GP, where the cleavage site is indicated by |. Thus, whether the affinity tag if fused to the N- or C-terminus of the protein of interest, either the “GP” or the “LEVLFQ” peptide sequence will remain attached to the protein of interest. One possibility is to fuse the protease site just upstream of the protein of interest such that the first methionine of the protein is immediately after the protease cleavage site. For instance, this would involve the configuration “Affinity tag-LEVLFQ|M . . . protein of interest” where the LEVLFQ| indicates the recognition site of the protease. However, a sequence with a methionine immediately after the cleavage site is very inefficiently cut by the protease, due to steric hindrance from the bulky methionine residue as well as the likely steric hindrance from the protein of interest itself.

Hence, there is need in the art for a protein purification system that allows the efficient and systematic purification of natives (non-modified) proteins.

### SUMMARY OF THE INVENTION

It is an object of the present invention to meet the above need by providing an isolated polypeptide comprising a protein of interest and an affinity tag, which allows the purification of the protein of interest on an affinity matrix. The protein of interest is further fused to a truncated protease recognition site, which is located directly adjacent to the N-terminus of the protein of interest and allows the release of the native protein of interest (this means without any additional amino acids) from an affinity matrix by a corresponding protease. However, the truncated protease recognition site only allows minimal or even no binding of the wild type protease to this site due to steric hindrance from the bulky methionine, whereby cleavage of the recognition site becomes inefficient.

Surprisingly, the present inventors have found that the inefficient binding of a protease to its truncated protease recognition site can be efficiently overcome by labeling each of (A) the protease and (B) the protein of interest fusion protein containing the protease recognition site with one member of a pair of binding partners resulting in enforced co-localization. The fusion to binding partners does not interfere with the activity of the protease and re-establishes sufficient cleavage activities.

In a first aspect, the present invention is thus directed to an isolated polypeptide comprising (A) a protein of interest; (B) a first member of a pair of binding partners; (C) an affinity tag for immobilizing the polypeptide on a solid support; and (D) a modified endoprotease recognition site, wherein the modified endoprotease site is located directly adjacent to the N-terminal amino acid of the protein of interest and comprises or only consists of the amino acid sequence N-terminal of the cleavage site of the native endoprotease recognition site.

In various embodiments of the invention, the first member of the pair of binding partners is located N-terminal to the modified protease recognition site and/or the affinity tag is located on the N- or C-terminus of the polypeptide, preferably the N-terminus.

The scope of the present invention also encompasses various embodiments wherein the polypeptide has in N- to C-terminal orientation the general formula (I) A-X-C-POI (I), wherein A represents the affinity tag; X represents the first member of the pair of binding partners; C represents the modified protease recognition site; POI represents the protein of interest; and “-” represents a peptide linker or peptide bond, wherein C and POI are linked by a peptide bond.

In still further various embodiments of the invention, the affinity tag is selected from the group consisting of a 6× His-tag, glutathione-S-transferase (GST) tag, chitin binding domain (CBD), calmodulin binding peptide (CBP), and maltose binding protein (MBP). In other various embodiments, the first member of the pair of binding partners is a peptide or polypeptide. In more preferred embodiments, the pair of binding partners is a pair of binding proteins or peptides. In even more preferred embodiments, the first member of a pair of binding partners is any member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrome (ARVCF) peptides (SEQ ID Nos. 7-9) as well as C-terminal fragments of the ARVCF peptides, and (iv) coiled coil (poly)peptide pairs.

Also encompassed by the scope of the present invention is that in various embodiments the modified endoprotease recognition site is derived from staphylococcall serine protease-like B (SplB) protease, human rhinovirus 3C (HRV3C) protease, tobacco etch virus (TEV) protease and tobacco vein mottling virus (TVMV) protease recognition sites.

In various embodiments, the modified endoprotease recognition site is derived from (1) an SplB protease recognition site and has the amino acid sequence WELQ (SEQ ID NO:1) or a derivative thereof; or (2) an HRV3C protease recognition site and has the amino acid sequence LEVLFQ (SEQ ID NO:2) or a derivative thereof; or (3) a TEV protease recognition site and has the amino acid sequence ENLYFQ (SEQ ID NO:3) or a derivative thereof; or (3) a TVMV protease recognition site and has the amino acid sequence ETVRFQ (SEQ ID NO:4) or a derivative thereof.

In further various embodiments of the invention, the derivatives of the modified endoprotease recognition sites comprise 1 or 2 amino acid substitutions relative to the amino acid sequences set forth in SEQ ID Nos. 1-4 and/or the N-terminal amino acid of the protein of interest is a methionine (M) residue.

In a further aspect, the present invention relates to a nucleic acid molecule encoding the polypeptide of the invention. In various embodiments, the nucleic acid molecule is comprised in a vector, preferably an expression vector.

In a still further aspect of the invention, the scope encompasses a host cell comprising the nucleic acid molecule of the invention.

In a fourth aspect, the invention relates to a method for isolating a protein of interest, comprising (a) expressing the protein of interest in form of a fusion protein according to the polypeptide of the invention as described above in a suitable expression system; (b) contacting the fusion protein obtained in step (a) with a protease fusion protein, wherein the protease fusion protein comprises a protease domain capable of recognizing and cleaving the modified protease recognition site and the second member of the pair of binding partners, under conditions that allow binding of the fusion protein and the protease fusion protein by binding of the pair of binding partners and cleavage of the modified protease recognition site, thereby releasing the protein of interest from the fusion protein; and (c) isolating the protein of interest.

In various embodiments of the method, the protease fusion protein further comprises an affinity tag identical to that of the fusion protein comprising the protein of interest. In other various embodiments, the fusion protein is expressed in a cellular expression system. In preferred embodiments, the fusion protein is expressed by cultivating the host cell of the invention under conditions that allow expression of the fusion protein. In various embodiments, prior to step (b) the expressed fusion protein is at least partially purified. In preferred embodiments, at least partial purification is carried out by subjecting the expressed fusion protein to affinity chromatography under conditions that allow immobilization of the fusion protein by interaction of the affinity tag with the solid affinity chromatography matrix. In more preferred embodiments, step (b) is carried out while the fusion protein is immobilized on an affinity chromatography material.

The scope of the present invention also encompasses various embodiments wherein step (c) comprises separating the cleaved protein of interest from the remainder of the fusion protein, preferably by eluting the released protein of interest from an affinity chromatography matrix on which the fusion protein has been immobilized. In various embodiments, the protease is SplB protease, HRV3C protease, TEV protease or TVMV protease. In further various embodiments of the invention, the second member of the pair of binding partners is a peptide or polypeptide. In preferred embodiments, the pair of binding partners is a pair of binding proteins or peptides.

Also encompassed by the scope of the present invention is that in various embodiments the second member of a pair of binding partners is the other member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrome (ARVCF) peptide (SEQ ID Nos. 7-9) as well as C-terminal fragments of the ARVCF peptides, and (iv) coiled coil (poly)peptide pairs.

In still further various embodiments, the invention relates to methods wherein the protease specifically recognizes and cleaves the modified protease recognition site. Further, (a) the fusion protein comprising the protein of interest or (b) the protein of interest do not comprise another site recognized and cleaved by the protease.

In a further aspect, the present invention relates to a kit for protein purification, comprising (a) an expression vector comprising a nucleic acid sequence encoding for an affinity tag, one member of a pair of binding partners and a modified endoprotease recognition site that allows generating a nucleic acid molecule according to the present invention by cloning a nucleic acid sequence encoding for a protein of interest into said expression vector; and (b) a protease fusion protein comprising a protease domain capable of recognizing and cleaving the modified protease recognition site and the other member of the pair of binding partners and optionally an affinity tag identical to that encoded by the expression vector.

### BRIEF DESCRIPTION OF THE DRAWINGS

The invention will be better understood with reference to the detailed description when considered in conjunction with the non-limiting examples and the accompanying drawings.

FIG. 1 shows schematic depictions of a protein of interest fusion peptide and a corresponding protease fusion peptide. Legend: (A) Schematic depiction of the target protein (brown) with a N-terminal fusion tag comprising a His-tag (yellow), binding protein X (green) and a protease site (blue) with the first methionine of the target protein at the P1′ position. (B) Schematic depiction of the protease (blue), binding Protein Y (purple) and His-tag (yellow).

FIG. 2 shows the process of protein purification. Legend: (A) The N-terminal tag-target protein fusion is bound to the affinity matrix. The His-tag is shown as a yellow line, binding protein X is the green rectangle, the protease site with the first methionine of the target protein in the P1′ position is the blue line, while the target protein is a brown oval. (B) The protease (blue 3/4^{th} circle) fused to binding protein Y (purple line) and a His-tag (yellow line) is added and binds to the target protein fusion via the binding protein X and Y interaction. (C) Owing to the binding of proteins X and Y, the close proximity of the protease to its recognition/cleavage site enables cleavage despite the suboptimal nature of this site. The free native target protein is eluted from the affinity matrix while the N-terminal fusion and the protease remain bound to the affinity matrix.

FIG. 3 shows the cleavage and purification results of a purification system composed according to the present invention using the lactamase Tem1.

FIG. 4 shows the cleavage and purification results of a purification system composed according to the present invention using LSSmOrange.

FIG. 5 shows the enhanced cleavage of a target fusion protein by enforced co-localization. Orange fluorescent protein (OFP) was expressed as a fusion with ePDZ-b connected by WELQ peptide substrate for SplB protease. 30 μg of this protein (ePDZ-b-WELQOFP) was incubated with varying amounts of the indicated SplB protease variants. These included SplB with full-length ARVC-pep tag at C-terminus (SplB-QPVDSWV) and 3 progressively shortened peptide tags. These tagged proteases all showed improved cleavage to yield native OFP (red arrow) compared to SplB protease tagged with a non-specific C-terminal peptide (SplB-CON) and commercial nontagged SplB protease (SplB-COM).

FIG. 6 shows improved cleavage of target fusion protein comprising TEV cleavage site with methionine at P1′ position. A) The ePDZ-b-ENLYFQ-OFP fusion protein (ENLYFQ is truncated consensus TEV recognition sequence) was incubated with either TEV protease tagged with optimized 4 amino acid ARVC-peptide (TEV-AP4) (lanes 2-9) or untagged TEV protease (lanes 13-20) for indicated times. Native OFP (arrowed red) was rapidly generated through use of TEV-AP4 compared to endogenous TEV. Lanes 11 and 22 show untreated fusion substrate. B) Same as in A, except using the fusion protein substrate MPB-ENLYFQS-PH-G1VCA with optimal TEV recognition sequence (underlined). Similar cleavage was observed for both TEV-AP4 (lanes 2-9) and endogenous TEV (lanes 14-21) to yield S-PHG1VCA. Lanes 12 and 24 respectively show TEV-AP4 and TEV proteases (dotted arrows). A lower molecular weight protein consistently co-purified with TEV. Lanes 11 and 23 show untreated fusion substrate.

FIG. 7 shows the improved on-column cleavage using imidazole-containing buffer. The HIS-ePDZ-b-WELQ-OFP fusion substrate protein and HIS-SplB-ARVC-pep were co-immobilized and on-column cleavage carried out overnight in buffer with (left gel) or without (right gel) imidazole. The results indicate improved cleavage and yields of native OFP in the presence of imidazole (compare “elution 1” lanes).

FIG. 8 shows the improved on-column cleavage of a recalcitrant fusion protein substrate by TEV-AP4. The HIS-ePDZ-b-ENLYFQ-OFP fusion substrate protein and either HISTEV-AP4 (left gel) or HIS-TEV (right gel) were co-immobilized and on-column cleavage carried out overnight. Lanes 2+11: Bacterial cell-lysate. Lanes 3-5/12-14: non-specific proteins eluted after three washes post loading. Lanes 6+15: native OFP (highlighted by asterisk) in flow-through post protease incubation. Lanes 7+16: Proteins eluted from column post-digestion by imidazole. Lanes 9+18: HIS-TEV-AP4 and HIS-TEV proteases.

FIG. 9 shows mass spectrometry analysis indicating generation of native OFP with N-terminal methionine upon cleavage of ePDZ-b-ENLYFQ-OFP substrate with TEVAP4 protease. Clear b and y ion series were identified (table below) corresponding to peptide sequences C-terminal to cleavage site with majority cleaved before N-terminal methionine of OFP.

FIG. 10 shows Edman degradation analysis shows prevalence of expected OFP N-terminal methionine upon cleavage of ePDZ-b-ENLYFQ-OFP substrate with TEVAP4 protease.

### DETAILED DESCRIPTION OF THE INVENTION

The present inventors surprisingly found that the decreased efficiency of a protease to bind to and cleave a peptide containing its shortened (truncated) protease recognition site can be overcome by labeling each of the protease and the peptide containing the recognition site with one member of a pair of binding partners. The interaction of the binding partners enforces co-localization of the protease and its suboptimal recognition site to re-establish efficient protease cleavage. This effect can be used in a protein purification system to purify native proteins that do not contain any additional amino acids compared to their natural amino acid sequence.

Therefore, in a first aspect, the present invention is thus directed to an isolated polypeptide comprising (A) a protein of interest; (B) a first member of a pair of binding partners; (C) an affinity tag for immobilizing the polypeptide on a solid support; and (D) a modified endoprotease recognition site, wherein the modified endoprotease site is located directly adjacent to the N-terminal amino acid of the protein of interest and comprises the amino acid sequence N-terminal of the cleavage site of the native endoprotease recognition site. In a different aspect, the invention relates to an isolated polypeptide comprising (A) a protein of interest; (B) a first member of a pair of binding partners; (C) an affinity tag for immobilizing the polypeptide on a solid support; and (D) a modified endoprotease recognition site, wherein the modified endoprotease site is located directly adjacent to the N-terminal amino acid of the protein of interest and only consists of the amino acid sequence N-terminal of the cleavage site of the native endoprotease recognition site.

The terms “polypeptide”, “protein”, and “peptide”, which are used interchangeably herein, refer to a polymer of the 20 protein amino acids, or amino acid analogs, regardless of the size or function of the molecule. Although “protein” is often used in reference to relatively large polypeptides, and “peptide” is often used in reference to small polypeptides, usage of these terms in the art overlaps and varies. Thus, the above terms relate to one or more associated molecules, wherein the molecules consist of amino acids coupled by peptide (amide) bonds. The amino acids are preferably the 20 naturally occurring amino acids glycine, alanine, valine, leucine, isoleucine, phenylalanine, cysteine, methionine, proline, serine, threonine, glutamine, asparagine, aspartic acid, glutamic acid, histidine, lysine, arginine, tyrosine and tryptophan.

The peptides and conjugates/fusion proteins of the invention can be synthesized synthetically or can be expressed in an organism or can be produced by in vitro transcription/translation. The peptides or conjugates may be expressed in, but such expression is not limited to Escherichia coli, Saccharomyces cerevisiae, Candida albicans, Pichia pastoris, insect cells such as Sf9 (Spodoptera frugiperda) cells, Nicotiana (tobacco plant) and CHO (Chinese hamster ovary) cells. Alternatively, the peptide or conjugate of the invention are expressed by an in vitro transcription/translation or “IVTT” system. “IVTT reaction” or “in vitro transcription translation reaction”, as interchangeably used herein, relates to cell-free systems that allow for specific transcription and translation by comprising macromolecular components (RNA polymerase, 70S or 80S ribosomes, tRNAs, aminoacyl-tRNA synthetases, initiation, elongation and termination factors, etc.) required for transcription and translation. To ensure efficient translation, the system may also be supplemented with amino acids, energy sources (ATP, GTP), energy regenerating systems, and other co-factors (Mg2+, K+, etc.). Such systems or extracts are also known as “coupled” and “linked” systems as they start with DNA templates, which are subsequently transcribed into RNA and then translated. Preferred IVTT reactions comprise the rabbit reticulocyte lysate, the wheat germ extract and the E. coli cell-free system.

Alternatively to the in vivo or in vitro expression of peptides, the synthesis of the peptide or conjugate of the invention is a synthetic synthesis. Methods of synthetic peptide synthesis include, but are not limited to liquid-phase peptide synthesis and solid-phase peptide synthesis (SPPS). Methods to produce peptides synthetically and according protocols are well-known in the art (Nilsson, BL et al. (2005) Annu Rev Biophys Biomol Struct, 34, 91). The synthesized peptides may be further modified by the attachment of additional chemical moieties.

Polypeptides referred to herein as “isolated” are polypeptides separated from other polypeptides and other cellular components of their source of origin (e.g., as it exists in cells or in an in vitro or synthetic expression system), and may have undergone further processing. “Isolated”, as used herein, refers to polypeptides or amino acid sequences that are at least 60% free, preferably 75% free, and most preferably 90% free from other components with which they are naturally associated. This percentage value may relate to the weight or the molarity of the polypeptide of the invention. “Isolated” polypeptides include polypeptides obtained by methods described herein, similar methods or other suitable methods, including essentially pure polypeptides, polypeptides produced by chemical synthesis, by combinations of biological and chemical methods, and recombinant polypeptides which are isolated. “Isolating”, as used herein, is defined as the process of releasing and obtaining a single constituent, such as a defined macromolecular species, from a mixture of constituents, such as from a culture of recombinant cells. This is typically accomplished by means such as centrifugation, filtration with or without vacuum, filtration under positive pressure, distillation, evaporation or a combination thereof. Isolating may or may not be accompanied by purifying during which the chemical, chiral or chemical and chiral purity of the isolate is increased. Purifying is typically conducted by means such as crystallization, distillation, extraction, filtration through acidic, basic or neutral alumina, filtration through acidic, basic or neutral charcoal, column chromatography on a column packed with a chiral stationary phase, filtration through a porous paper, plastic or glass barrier, column chromatography on silica gel, ion exchange chromatography, recrystallization, normal-phase high performance liquid chromatography, reverse-phase high performance liquid chromatography, trituration and the like.

The term “protein of interest”, as used herein refers to any target protein, production thereof and optionally its modification, such as phosphorylation, glycosylation, acetylation, ADP-ribosylation, ubiquitilation and SUMOylation. In various embodiments, the protein of interest is an antibody or an antigen-binding fragment thereof, a soluble protein, a membrane protein, a structural protein, a ribosomal protein, an enzyme, a zymogen, a cell surface receptor protein, a transcription regulatory protein, a translation regulatory protein, a chromatin protein, a hormone, a cell cycle regulatory protein, a G-protein, a neuroactive peptide, an immunoregulatory protein, a blood component protein, an ion gate protein, a heat shock protein, an antibiotic resistance protein, a functional fragment of any of the preceding proteins, an epitope-containing fragment of any of the preceding proteins and combinations thereof. In a particular embodiment, the protein of interest is a monomer.

Generally, any peptide or protein may be chosen as a peptide of interest (PeOI) or a protein of interest (PrOI). In certain embodiments, the PrOI is a protein which does not form a homo-dimer or homo-multimer. The avoidance of self-interacting peptides or proteins may be advantageous if the recombinant peptide or protein is to be secreted into the cell culture supernatant, because the formation of larger protein complexes may disturb an efficient protein export. However, the PrOI may also be a peptide or protein, which is a subunit of a larger peptide or protein complex. Such a peptide or protein may be isolated after expression and optionally secretion and be suitable for an in vitro reconstitution of the multi peptide or protein complex. In certain embodiments, the PeOI or PrOI is a peptide having less than 100 amino acid residues. If these peptides comprise pre-and/or pro-sequences in their native state after translation the nucleic acid sequence encoding for the PeOI may be engineered to be limited to the sequence encoding the mature peptide. One exemplary peptide is insulin, e.g., human insulin.

In various embodiments, the PeOI or PrOI is an enzyme.

The International Union of Biochemistry and Molecular Biology has developed a nomenclature for enzymes, the EC numbers; each enzyme is described by a sequence of four numbers preceded by “EC”. The first number broadly classifies the enzyme based on its mechanism.

The complete nomenclature can be browsed at http://www.chem.qmul.ac.uk/iubmb/ienzyme/.

Accordingly, a PeOI or PrOI according to the present invention may be chosen from any of the classes EC 1 (Oxidoreductases), EC 2 (Transferases), EC 3 (Hydrolases), EC 4 (Lyases), EC 5 (Isomerases), and EC 6 (Ligases), and the subclasses thereof.

In certain embodiments, the PeOI or PrOI is cofactor dependent or harbors a prosthetic group. For expression of such peptides or proteins, in some embodiments, the corresponding cofactor or prosthetic group may be added to the culture medium during expression.

In certain cases, the PeOI or PrOI is a dehydrogenase or an oxidase.

In case the PeOI or PrOI is a dehydrogenase, in some embodiments, the PeOI or PrOI is chosen from the group consisting of alcohol dehydrogenases, glutamate dehydrogenases, lactate dehyrogenases, cellobiose dehydrogenases, formate dehydrogenases, and aldehydes dehydrogenases.

In case the PeOI or PrOI is an oxidase, in some embodiments, the PeOI or PrOI is chosen from the group consisting of cytochrome P450 oxidoreductases, in particular P450 BM3 and mutants thereof, peroxidases, monooxygenases, hydrogenases, monoamine oxidases, aldehydes oxidases, xanthin oxidases, amino acid oxidases, and NADH oxidases.

In further embodiments, the PeOI or PrOI is a transaminase or a kinase.

In case the PeOI or PrOI is a transaminase, in some embodiments, the PeOI or PrOI is chosen from the group consisting of alanine aminotransferases, aspartate aminotransferases, glutamate-oxaloacetic transaminases, histidinol-phosphate transaminases, and histidinol-pyruvate transaminases.

In various embodiments, if the PeOI or PrOI is a kinase, the PeOI or PrOI is chosen from the group consisting of nucleoside diphosphate kinases, nucleoside monophosphate kinases, pyruvate kinase, and glucokinases.

In some embodiments, if the PeOI or PrOI is a hydrolase, the PeOI or PrOI is chosen from the group consisting of lipases, amylases, proteases, cellulases, nitrile hydrolases, halogenases, phospholipases, and esterases.

In certain embodiments, if the PeOI or PrOI is a lyase, the PeOI or PrOI is chosen from the group consisting of aldolases, e.g., hydroxynitrile lyases, thiamine-dependent enzymes, e.g., benzaldehyde lyases, and pyruvate decarboxylases.

In various embodiments, if the PeOI or PrOI is an isomerase, the PeOI or PrOI is chosen from the group consisting of isomerases and mutases.

In some embodiments, if the PeOI or PrOI is a ligase, the PeOI or PrOI may be a DNA ligase.

In certain embodiments, the PeOI or PrOI may be an antibody. This may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like.

Also contemplated herein are therapeutically active PeOIs and PrOI, e.g., a cytokine.

Thus, in certain embodiments the PeOI or PrOI is selected from the group consisting cytokines, in particular human or murine interferons, interleukins, colony-stimulating factors, necrosis factors, e.g., tumor necrosis factor, and growth factors.

In some embodiments, if the PeOI or PrOI is an interferon, the PeOI or PrOI may be selected from the group consisting of interferon alpha, e.g., alpha-1, alpha-2, alpha-2a, and alpha-2b, alpha-2, alpha-8, alpha-16, alpha 21, beta, e.g., beta-1, beta-1a, and beta-1b, or gamma.

In further embodiments, the PeOI or PrOI is an antimicrobial peptide, in particular a peptide selected from the group consisting of bacteriocines and lantibiotics, e.g., nisin, cathelicidins, defensins, and saposins.

In further embodiments, the PeOI or PrOI is an adhesive peptide with distinct surface specificities, for example for steel, aluminum and other metals or specificities towards other surfaces like carbon, ceramic, minerals, plastics, wood and other materials or other biological materials like cells, or adhesive peptides that function in aqueous environments and under anaerobe conditions.

In further embodiments, the PeOI or PrOI has a length ranging from 2-100 amino acids, wherein said amino acids are selected from the group of the 20 proteinogenic amino acids.

“Binding pair” or “specific binding pair”, as interchangeably used herein, refers to two compounds that specifically bind to one another, such as (functionally): a receptor and a ligand (such as a drug), an antibody and an antigen, etc.; or (structurally): protein or peptide and protein or peptide; protein or peptide and nucleic acid; and nucleotide and nucleotide etc. In preferred embodiments the members of the binding pair directly bind to each other. Alternatively, in other preferred embodiments of the invention, the members of the binding pair are not binding by direct contact to each other. In these cases, the interaction of the members of the binding pair is “linked” or “bridged” by one or more linker molecules. “Specific binding pair” include, but are not limited to antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, and virus-receptor interactions. The “first member” of a binding pair can be any one of the two members independent of their structural position within the binding complex or other parameters defined by the given binding pair.

The term “affinity tag”, as used herein, refers to an amino acid sequence that is used to facilitate purification of a protein or polypeptide. In one embodiment, the affinity tag includes a streptavidin tag, a c-myc tag, an HA-tag, a T7 tag, a FLAG-tag, a polyhistidine tag (such as (His)₆), a polyarginine tag, a polyphenylalanine tag, a polycysteine tag, or a polyaspartic acid tag. In a specific embodiment, the affinity tag is (His)₆. The term “(His)₆”, as used herein, refers to the following amino acid sequence: HHHHHH. “Tag”, as used herein, may also relate to a group of atoms or a molecule that is attached covalently to a polypeptide or another biological molecule for the purpose of detection by an appropriate detection system. The term “tagged peptide” refers to a peptide to which a tag has been covalently attached. The term “tag” and “label” may be used interchangeably. The term “affinity chromatography”, as used herein, relates to the complex formation of the tagged peptide or protein and the receptor. In certain embodiments affinity tags may be selected from the group consisting of the Strep-tag® or Strep-tag® II, the myc-tag, the FLAG-tag, the His-tag, the small ubiquitin-like modifier (SUMO) tag, the covalent yet dissociable NorpD peptide (CYD) tag, the heavy chain of protein C (HPC) tag, the calmodulin binding peptide (CBP) tag, or the HA-tag or proteins such as Streptavidin binding protein (SBP), maltose binding protein (MBP), and glutathione-S-transferase. The term “solid support”, as used herein, refers to a solid or insoluble support, commonly a polymeric support, to which a linker moiety (that allows binding of the affinity tag) can be covalently bonded by reaction with a functional group of the support. Many suitable supports are known, and include materials such as polystyrene resins, polystyrene/divinylbenzene copolymers, agarose, and other materials known to the skilled person skilled in the art. It will be understood that an insoluble support can be soluble under certain conditions and insoluble under other conditions; however, for purposes of this invention, a polymeric support is “insoluble” if the support is insoluble or can be made insoluble in a reaction solvent. Further, the solid support may be a soluble or insoluble polymeric structure, such as polystyrene, or an inorganic structure, e.g. of silica or alumina

“Protease recognition site” or “endoprotease recognition site”, as interchangeably used herein, refer to a specific amino acid sequence that is recognized by a specific protease which subsequently cleaves the polypeptide by way of hydrolysis of an amide bond marked by the protease recognition site. Usually, the cleavage occurs within the recognition site. Thus, the recognition site can be separated into two different parts. One part of the recognition site, which is located N-terminal of the cleavage site of the protease and another one, which is located C-terminal of the cleavage site. The polypeptide of the present invention only comprises the amino acid sequence of the protease recognition site that is located N-terminal of the cleavage site of the native endoprotease. In preferred embodiments of the invention, the protease recognition site is a conserved motif that contains an N-terminal and a C-terminal part located around the cleavage site. In these embodiments, proteases, such as trypsin, are excluded which cleave peptides directly adjacent behind a short motif, such as a basic amino acid or a modified cysteine. In other various embodiments of the invention, the modified protease recognition site (meaning the complete or partial amino acid sequence of a conserved recognition motif that is located N-terminal of the cleavage site) comprises or consists of at least 2, 3, 4, 5, 6 or 7 amino acids. In other various embodiments, the modified protease recognition site comprises or consists of at most 15, 10, 9, 8, 7, 6, 5 or 4 amino acids. In preferred embodiments, the protease recognition site is a recognition site for an externally added protease, meaning that this protease does not occur or is not active in the organism, which expresses the polypeptide of the invention. The term “protease cleavage site” or “protease recognition site”, as interchangeably used herein, refers to a peptide sequence which can be cleaved by a selected protease thus allowing the separation of peptide or protein sequences which are interconnected by a protease cleavage site. In certain embodiments the protease cleavage site is selected from the group consisting of a Factor Xa-, a tobacco edge virus (TEV) protease-, a enterokinase-, a SUMO Express protease-, an IgA-Protease-, an Arg-C proteinase-, an Asp-N endopeptidases-, an Asp-N endopeptidase+N-terminal Glu-, a caspase1-, a caspase2-,a caspase3-, a caspase4, a caspase5, a caspase6, a caspase7, a caspase8, a caspase9, a caspase10, a chymotrypsin-high specificity, a chymotrypsin-low specificity-, a clostripain (Clostridiopeptidase B)-, a glutamyl endopeptidase-, a granzymeB-, a pepsin-, a proline-endopeptidase-, a proteinase K-, a staphylococcal peptidase I-, a Thrombin-, a Trypsin-, and a Thermolysin-cleavage site.

The term “directly adjacent”, as used herein, refers to adjacent amino acid sequence fragments of the polypeptide of the invention, in particular the protein of interest and the modified endoprotease recognition site, that are in contact with each other without any other amino acid sequence therebetween. Based on the subject-matter of the present invention, this means that the most C-terminal amino acid of the modified endoprotease recognition site directly precedes the most N-terminal amino acid of the protein of interest. Thus, if the amino acid sequence of the endoprotease recognition site is “LEVLFQ” and the amino acid sequence of the protein of interest starts with a “M”, then the polypeptide of the invention inevitable comprises the sequence “LEVLFQM”.

In various embodiments of the invention, the first member of the pair of binding partners is located N-terminal to the modified protease recognition site and/or the affinity tag is located on the N- or C-terminus of the polypeptide, preferably the N-terminus.

The term “N-terminus” relates to the start of a protein or polypeptide, terminated by an amino acid with a free amine group (—NH2).

The term “an N-terminal fragment” relates to a peptide or protein sequence which is in comparison to a reference peptide or protein sequence C-terminally truncated, such that a contiguous amino acid polymer starting from the N-terminus of the peptide or protein remains. In some embodiments, such fragments may have a length of at least 10, 20, 50, or 100 amino acids.

The term “C-terminus” relates to the end of an amino acid chain (protein or polypeptide), terminated by a free carboxyl group (—COOH).

The term “a C-terminal fragment” relates to a peptide or protein sequence which is in comparison to a reference peptide or protein sequence N-terminally truncated, such that a contiguous amino acid polymer starting from the C-terminus of the peptide or protein remains. In some embodiments, such fragments may have a length of at least 10, 20, 50, or 100 amino acids.

“At least one”, as used herein, relates to one or more, in particular 1, 2, 3, 4, 5, 6, 7, 8, 9, 10 or more.

The scope of the present invention also encompasses various embodiments wherein the polypeptide has in N- to C-terminal orientation the general formula (I) A-X-C-POI (I), wherein A represents the affinity tag; X represents the first member of the pair of binding partners; C represents the modified protease recognition site; POI represents the protein of interest; and “-” represents a peptide linker or peptide bond, wherein C and POI are linked by a peptide bond. The term “peptide linker”, as used herein, refers to a sequence of amino acids, preferably 1 to 20 amino acids, which are linearly linked to each other by peptide bonding. The peptide linker may be modified, but with respect to the present objects, it is preferably non-modified. The term “peptide bond”, as used herein, includes reference to a covalent chemical bond formed between two amino acids when the carboxylic acid group of one molecule reacts with the amino group of the other molecule. In certain embodiments, the PeOI or PrOI comprises a deletion of at least 10, 20, 30, 40, 50, or more N- and/or C-terminal amino acid relative to the wildtype peptide or protein sequence.

In still further various embodiments of the invention, the affinity tag is selected from the group consisting of a 6× His-tag, glutathione-S-transferase (GST) tag, chitin binding domain (CBD), calmodulin binding peptide (CBP), and maltose binding protein (MBP). In other various embodiments, the first member of the pair of binding partners is a peptide or polypeptide. In more preferred embodiments, the pair of binding partners is a pair of binding proteins or peptides. In even more preferred embodiments, the first member of a pair of binding partners is any member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrome (ARVCF) peptides (SEQ ID Nos. 7-9) as well as C-terminal fragments of the ARVCF peptides, and (iv) coiled coil (poly)peptide pairs.

The term “small peptide”, as used herein, refers to a peptide consisting of at most 25, 20, 15, 14, 13, 12, 11, 10, 9, 8, 7, 6, 5 amino acids. The term “small molecule”, as used herein, refers to molecules according to Lipinski's rule of five. The term “aptamer”, as used herein, refers to a single-stranded oligonucleotide (single-stranded DNA or RNA molecule) that can bind specifically to its target with high affinity. Particularly, aptamers can be used as molecules targeting various organic and inorganic materials, including toxins, unlike antibodies. The advantages and structural properties of aptamers are described by Kim and Man-Bock (Yeon-Seok, Kim and Man-Bock, Gu, 2008, NICE, 26(6):690). The term “split domain” relates to a protein domain that is split into two parts that bind to each other to re-assemble the complete domain. In various embodiments, the split domains are peptides as set forth in SEQ ID Nos. 5 and 6, which allow re-constitution of the FbaB-type fibronectin-binding protein of Streptococcus pyrogenes. “Functional fragment or derivative”, as used herein, is a peptide or polypeptide, optionally carrying one or more post-translational modifications, which, when compared to the non-modified full-length member of the binding pair, provides similar binding properties as the non-modified member. In various embodiments, the functional fragment or derivative has at least 70%, 75%, 80%, 85%, 90%, 95% or 98% of the binding capacity of the non-modified first member towards the second member of the binding pair. In various other embodiments of the invention, the functional fragment or derivative has at least 70%, 75%, 80%, 85%, 90%, 95% or 98% sequence homology to a first member of a given binding pair measured over the whole length of the amino acid sequence of the first member. “Coil-coil” or “coiled coil”, as used herein, refers to an α-helical oligomerization domain found in a variety of proteins. Proteins with heterologous domains joined by coiled coils are described in U.S. Pat. Nos. 5,716,805 and 5,837,816. Structural features of coiled-coils are described in Litowski and Hodges, J. Biol. Chem. 277:37272-27279, 2002; Lupas TIBS 21:375-382 (1996); Kohn and Hodges TIBTECH16: 379-389(1998); and Müller et al. Methods Enzymol. 328: 261-282 (2000). Coiled-coils generally comprise two to five α-helices (see, e.g., Litowski and Hodges, 2002, supra). The α-helices may be the same or difference and may be parallel or anti-parallel. Typically, coiled-coils comprise an amino acid heptad repeat: “abcdefg”.

Also encompassed by the scope of the present invention is that in various embodiments the modified endoprotease recognition site is derived from staphylococcal serine protease-like B (SplB) protease, human rhinovirus 3C (HRV3C) protease, tobacco etch virus (TEV) protease and tobacco vein mottling virus (TVMV) protease recognition sites.

In various embodiments, the modified endoprotease recognition site is derived from (1) an SplB protease recognition site and has the amino acid sequence WELQ (SEQ ID NO:1) or a derivative thereof; or (2) an HRV3C protease recognition site and has the amino acid sequence LEVLFQ (SEQ ID NO:2) or a derivative thereof; or (3) a TEV protease recognition site and has the amino acid sequence ENLYFQ (SEQ ID NO:3) or a derivative thereof; or (3) a TVMV protease recognition site and has the amino acid sequence ETVRFQ (SEQ ID NO:4) or a derivative thereof.

In further various embodiments of the invention, the derivatives of the modified endoprotease recognition sites comprise 1 or 2 amino acid substitutions relative to the amino acid sequences set forth in SEQ ID Nos. 1-4 and/or the N-terminal amino acid of the protein of interest is a methionine (M) residue.

In a further aspect, the present invention relates to a nucleic acid molecule encoding the polypeptide of the invention. In various embodiments, the nucleic acid molecule is comprised in a vector, preferably an expression vector.

The term “nucleic acid molecule” or “nucleic acid sequence”, as used herein, relates to DNA (deoxyribonucleic acid) or RNA (ribonucleic acid) molecules. Said molecules may appear independent of their natural genetic context and/or background. The term “nucleic acid molecule/sequence” further refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms.

The polypeptide of the invention may be cloned into a vector. In certain embodiments, the vector is selected from the group consisting of a pSU-vector, pET-vector, a pBAD-vector, a pK184-vector, a pMONO-vector, a pSELECT-vector, pSELECT-Tag-vector, a pVITRO-vector, a pVIVO-vector, a pORF-vector, a pBLAST-vector, a pUNO-vector, a pDUO-vector, a pZERO-vector, a pDeNy-vector, a pDRIVE-vector, a pDRIVE-SEAP-vector, a HaloTag®Fusion-vector, a pTARGET™-vector, a Flexi®-vector, a pDEST-vector, a pHIL-vector, a pPIC-vector, a pMET-vector, a pPink-vector, a pLP-vector, a pTOPO-vector, a pBud-vector, a pCEP-vector, a pCMV-vector, a pDisplay-vector, a pEF-vector, a pFL-vector, a pFRT-vector, a pFastB ac-vector, a pGAPZ-vector, a pIZ/V5-vector, a pLenti6-vector, a pMIB-vector, a pOG-vector, a pOpti-vector, a pREP4-vector, a pRSET-vector, a pSCREEN-vector, a pSecTag-vector, a pTEF1-vector, a pTracer-vector, a pTrc-vector, a pUB6-vector, a pVAX1-vector, a pYC2-vector, a pYES2-vector, a pZeo-vector, a pcDNA-vector, a pFLAG-vector, a pTAC-vector, a pT7-vector, a gateway®-vector, a pQE-vector, a pLEXY-vector, a pRNA-vector, a pPK-vector, a pUMVC-vector, a pLIVE-vector, a pCRUZ-vector, a Duet-vector, and other vectors or derivatives thereof.

The vectors of the present invention may be chosen from the group consisting of high, medium and low copy vectors.

The above described vectors may be used for the transformation or transfection of a host cell in order to achieve expression of a peptide or protein which is encoded by an above described nucleic acid molecule and comprised in the vector DNA.

In a still further aspect of the invention, the scope encompasses a host cell comprising the nucleic acid molecule of the invention.

The term “host cell”, as used herein, relates to an organism that harbors the nucleic acid molecule or a vector encoding the polypeptide of the invention. In preferred embodiments the host cell is a prokaryotic cell. In more preferred embodiments the host cell is E. coli which may include but is not limited to BL21, DH1, DH5α, DM1, HB101, JM101-110, K12, Rosetta(DE3)pLysS, SURE, TOP10, XL1-Blue, XL2-Blue and XL10-Blue strains.

The host cell may be specifically chosen as a host cell capable of expressing the gene. In addition or otherwise, in order to produce a peptide or protein, a fragment of the peptide or protein or a fusion protein of the peptide or protein with another polypeptide, the nucleic acid coding for the peptide or protein can be genetically engineered for expression in a suitable system. Transformation can be performed using standard techniques (Sambrook, J. et al. (2001), supra).

Prokaryotic or eukaryotic host organisms comprising such a vector for recombinant expression of the polypeptide of the invention as described herein form also part of the present invention. Suitable host cells can be prokaryotic cell. In certain embodiments the host cells are selected from the group consisting of gram positive and gram negative bacteria. In some embodiments, the host cell is a gram negative bacterium, such as E. coli. In certain embodiments, the host cell is E. coli, in particular E. coli BL21 (DE3) or other E. coli K12 or E. coli B834 or E. coli DH5a or XL-1 derivatives. In further embodiments, the host cell is selected from the group consisting of Escherichia coli (E. coli), Pseudomonas, Serratia marcescens, Salmonella, Shigella (and other enterobacteriaceae), Neisseria, Hemophilus, Klebsiella, Proteus, Enterobacter, Helicobacter, Acinetobacter, Moraxella, Helicobacter, Stenotrophomonas, Bdellovibrio, Legionella, acetic acid bacteria, Bacillus, Bacilli, Carynebacterium, Clostridium, Listeria, Streptococcus, Staphylococcus, and Archaea cells. Suitable eukaryotic host cells are among others CHO cells, insect cells, fungi, yeast cells, e.g., Saccharomyces cerevisiae, S. pombe, Pichia pastoris.

The transformed host cells are cultured under conditions suitable for expression of the nucleotide sequence encoding a peptide or protein of the invention. In certain embodiments, the cells are cultured under conditions suitable for expression of the nucleotide sequence encoding the polypeptide of the invention.

For producing the polypeptide of the invention, a vector may be introduced into a suitable prokaryotic or eukaryotic host organism by means of recombinant DNA technology. For this purpose, the host cell is first transformed with a vector comprising a nucleic acid molecule according to the present invention using established standard methods (Sambrook, J. et al. (2001), supra). The host cell is then cultured under conditions, which allow expression of the heterologous DNA and thus the synthesis of the corresponding polypeptide. Subsequently, the polypeptide is recovered either from the cell.

For expression of the peptides and proteins of the present invention several suitable protocols are known to the skilled person.

Generally, any known culture medium suitable for growth of the selected host may be employed in this method. In various embodiments, the medium is a rich medium or a minimal medium. Also contemplated herein is a method, wherein the steps of growing the cells and expressing the peptide or protein comprise the use of different media. For example, the growth step may be performed using a rich medium, which is replaced by a minimal medium in the expression step. In certain cases, the medium is selected from the group consisting of LB medium, TB medium, 2YT medium, synthetical medium and minimal medium.

In some embodiments, the medium may be supplemented with IPTG, arabinose, tryptophan and/or maltose, and/or the culture temperature may be changed and/or the culture may be exposed to UV light. In various embodiments, the conditions that allow secretion of the recombinant peptide or protein are the same used for the expression of the peptide or protein.

In certain embodiments, the host cell is a prokaryotic cell, such as E. coli, in particular E. coli BL21 (DE3) and E. coli DH5α.

In some embodiments, the entire culture of the host cell, e.g., during growth and expression, is carried out in minimal medium. Minimal medium is advantageous for recombinant peptide or protein expression, as the protein, lipid, carbohydrate, pigment, and impurity content in this medium is reduced and thus circumvents or reduces the need of extensive purification steps.

In a fourth aspect, the invention relates to a method for isolating a protein of interest, comprising (a) expressing the protein of interest in form of a fusion protein according to the polypeptide of the invention as described above in a suitable expression system; (b) contacting the fusion protein obtained in step (a) with a protease fusion protein, wherein the protease fusion protein comprises a protease domain capable of recognizing and cleaving the modified protease recognition site and the second member of the pair of binding partners, under conditions that allow binding of the fusion protein and the protease fusion protein by binding of the pair of binding partners and cleavage of the modified protease recognition site, thereby releasing the protein of interest from the fusion protein; and (c) isolating the protein of interest.

The terms “expression” or “expressed”, as interchangeably used herein, relate to a process in which information from a gene is used for the synthesis of a gene product, usually a polypeptide or protein. In cell-based expression systems the expression comprises transcription and translation steps.

The term “fusion protein”, as used herein, generally indicates a polypeptide in which heterogenous polypeptides having different origins are linked, and in the present invention, refers to (a) a polypeptide in which the above described peptide fragments are linked to result in the polypeptide of the invention and (b) a protease able to cleave a modified recognition site linked to a second member of a binding pair.

“Culturing”, “cultivating” or “cultivation”, as used herein, relates to the growth of a host cell in a specially prepared culture medium under supervised conditions. The terms “conditions suitable for recombinant expression” or “conditions that allow expression” relate to conditions that allow for production of the polypeptide of the invention in host cells using methods known in the art, wherein the cells are cultivated under defined media and temperature conditions. The medium may be a nutrient, minimal, selective, differential, or enriched medium. Preferably, the medium is a minimal culture medium. Growth and expression temperature of the host cell may range from 4° C. to 45° C. Preferably, the growth and expression temperature range from 30° C. to 39° C. The term “expression medium” as used herein relates to any of the above media when they are used for cultivation of a host cell during expression of a protein.

The term “contacting”, as used herein, refers generally to providing access of one component, reagent, analyte or sample to another. For example, contacting can involve mixing a solution comprising the polypeptide of the invention with a protease fusion protein. The solution comprising one component, reagent, analyte or sample may also comprise another component or reagent, such as dimethyl sulfoxide (DMSO) or a detergent, which facilitates mixing, interaction, uptake, or other physical or chemical phenomenon advantageous to the contact between components, reagents, analytes and/or samples.

The terms “binding”, “specifically bind” and “specific binding”, as interchangeably used herein, generally refer to the ability of a first given molecule to preferentially bind to a second molecule, which may be the same or different type than the first molecule, that is present in a homogeneous mixture of different molecules. In certain embodiments, a specific binding interaction will discriminate between desirable and undesirable antigens in a sample, in some embodiments more than about 10 to 100-fold or more (e.g., more than about 1000- or 10,000-fold). The term “conditions that allow binding” refers to a combination of different parameters, such as temperature, pH value, salt and detergent concentrations, that allow the binding of a given first molecule to a second molecule. With respect to well-established binding pairs such conditions are usually well-known by the person skilled in the art.

The term “releasing”, as used herein with regard to the protein of interest, means that the polypeptide of the invention is cleaved by a protease fusion protein to obtain two “free” (separated) proteins. The cleavage of the polypeptide of the invention results in a “free” protein of interest and a second polypeptide comprising the remaining sections of the polypeptide of the invention. In various embodiments, the polypeptide of the invention is dissolved in a solvent prior to the cleavage of the protease. In these cases, the protein of interest and the remaining polypeptide dissociate after cleavage due to natural thermodynamic dissociation. In alternative embodiments, the polypeptide is attached to an affinity matrix prior cleavage. In these cases, after cleavage the remaining polypeptide still attaches to the affinity matrix, while the protein of interest is solved in the solvent and dissociates from the affinity matrix.

In various embodiments of the method, the protease fusion protein further comprises an affinity tag identical to that of the fusion protein comprising the protein of interest. In other various embodiments, the fusion protein is expressed in a cellular expression system. In preferred embodiments, the fusion protein is expressed by cultivating the host cell of the invention under conditions that allow expression of the fusion protein. In various embodiments, prior to step (b) the expressed fusion protein is at least partially purified. In preferred embodiments, the at least partial purification is carried out by subjecting the expressed fusion protein to affinity chromatography under conditions that allow immobilization of the fusion protein by interaction of the affinity tag with the solid affinity chromatography matrix. In more preferred embodiments, step (b) is carried out while the fusion protein is immobilized on an affinity chromatography material.

A “cellular expression system”, as used herein, comprises prokaryotic and eukaryotic organism, such as bacterial, plant, fungus or animal cells and cell cultures derived thereof.

The term “partially purified”, as used herein, relates to a molecule, in particular the polypeptide of the invention, that is at least 60% free, preferably 75% free, and most preferably 90% free from other components with which it is naturally associated or which are used for the synthesis of the polypeptide of the present invention. These percentage values may relate to the weight or the molarity of the polypeptide of the invention.

The scope of the present invention also encompasses various embodiments wherein step (c) comprises separating the cleaved protein of interest from the remainder of the fusion protein, preferably by eluting the released protein of interest from an affinity chromatography matrix on which the fusion protein has been immobilized. In various embodiments, the protease is SplB protease, HRV3C protease, TEV protease or TVMV protease. In further various embodiments of the invention, the second member of the pair of binding partners is a peptide or polypeptide. In preferred embodiments, the pair of binding partners is a pair of binding proteins or peptides.

Also encompassed by the scope of the present invention is that in various embodiments the second member of a pair of binding partners is the other member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrom (ARVCF) peptide (SEQ ID Nos. 7-9) as well as C-terminal fragments of the ARVCF peptides, and (iv) coiled coil (poly)peptide pairs.

In still further various embodiments, the invention relates to methods wherein the protease specifically recognizes and cleaves the modified protease recognition site. Further, (a) the fusion protein comprising the protein of interest or (b) the protein of interest do not comprise another site recognized and cleaved by the protease.

In a further aspect, the present invention relates to a kit for protein purification, comprising (a) an expression vector comprising a nucleic acid sequence encoding for an affinity tag, one member of a pair of binding partners and a modified endoprotease recognition site that allows generating a nucleic acid molecule according to the present invention by cloning a nucleic acid sequence encoding for a protein of interest into said expression vector; and (b) a protease fusion protein comprising a protease domain capable of recognizing and cleaving the modified protease recognition site and the other member of the pair of binding partners and optionally an affinity tag identical to that encoded by the expression vector.

The term “kit”, as used herein, relates to packaged reagents for protein purification. Accordingly, the kits of the invention comprise an expression vector encoding the polypeptide of the invention and a protease fusion protein. Additionally, such a kit may comprise instructions for use as well as typical reagents known to those skilled in the art.

The term “sequence”, as used herein, relates to the primary nucleotide sequence of nucleic acid molecules or the primary amino acid sequence of a protein.

As used herein, “sequence identity” or “identity” in the context of two nucleic acid or peptide sequences makes reference to the residues in the two sequences that are the same position when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well-known in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “entire length”, as used herein in the context of sequence identity, relates to the primary amino acid sequence of a given peptide ranging from the first amino acid at the N-terminus to the last amino acid at the C-terminus of said given peptide.

The term “conjugate”, as used herein, refers to a compound comprising two or more molecules (e.g., peptides, carbohydrates, small molecules, or nucleic acid molecules) that are chemically linked. The two or molecules desirably are chemically linked using any suitable chemical bond (e.g., covalent bond). Suitable chemical bonds are well known in the art and include disulfide bonds, acid labile bonds, photolabile bonds, peptidase labile bonds (e.g. peptide bonds), thioether, and esterase labile bonds.

In another aspect, the present invention relates to an isolated polypeptide comprising a (a) protein of interest and (b) an amino acid sequence as set forth in SEQ ID NO:10 or SEQ ID NO:11. In various embodiments, the protein of interest is a protease.

Further, the present invention is directed to a method for degrading a target protein, comprising providing a fusion protease protein, wherein the fusion protease protein comprises (a) a protease and (b) a target protein binding element, contacting the fusion protease protein with the target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a), wherein the target protein is degraded upon enforced interaction of the fusion protease protein and the target protein. In various embodiments, the target protein binding element is selected from the group consisting of a peptide, an antibody or a fragment thereof, an aptamer and a small molecule.

In a further aspect, the invention relates to a method for treatment of a disease, wherein a pathogenic target protein is degraded by a fusion protease protein, the method comprising providing the fusion protease protein, wherein the fusion protease protein comprises (a) a protease and (b) a target protein binding element, contacting the fusion protease protein with the pathogenic target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a), wherein the target protein is degraded upon enforced interaction of the fusion protease protein and the target protein.

In a still further aspect, the present invention is directed to a fusion protease protein for use as a medicament, wherein a pathogenic target protein is degraded by a fusion protease protein, the method comprising providing the fusion protease protein, wherein the fusion protease protein comprises (a) a protease and (b) a target protein binding element, contacting the fusion protease protein with the pathogenic target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a), wherein the target protein is degraded upon enforced interaction of the fusion protease protein and the target protein. The at least one amino acid sequence that has 40%-5490% homology over the whole length to a recognition site of the protease of (a) has in other various embodiments of the invention at least 45%, 50%, 55%, 60%, 65%, 70%, 75%, 80% or 85% homology over the whole length to a recognition site of the protease of (a). In other various embodiments, the homology over the whole length to a recognition site of the protease of (a) is at most 85%, 80%, 75%, 70%, 65%, 60%, 55%, 50% or 45%.

The term “pathogenic target protein”, as used herein, is used in the broad sense of an infectious protein and/or a simple product of disease. These protein include, but are not limited to oncogenes, prion protein (PrP^{Sc}), APP (Alzheimer's disease), 1-antichymotrypsin (Alzheimer's disease), tan (Alzheimer's disease), SOD (ALS), neurofilament (ALS), Pick body (Pick's disease), Lewy body (Parkinson's disease), Amylin (Diabetes Type 1), IgGL-chain (Multiple myeloma—plasma cell dyscrasias), Transthyretin (Familial amyloidotic polyneuropathy), Procalcitonin (Medulla carcinoma of thyroid), beta-2-microglobulin (Chronic renal failure), atrial natriuretic factor (congestive heart failure), serum amyloid A (chronic inflammation), ApoA1 (atherosclerosis) and Gelsolin (Familial amyloidosis).

### EXAMPLES

### Example 1

### Description of the Present Technology and Cleavage Results Using Two Different Recognition Site/Protease Systems

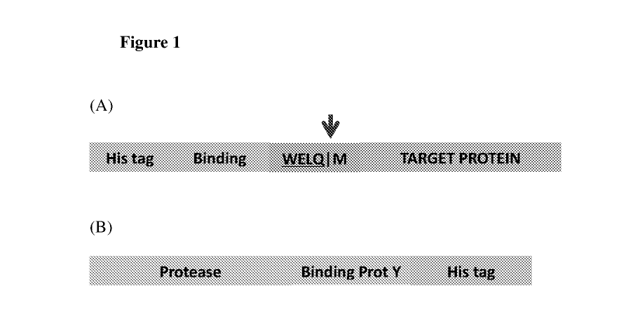

The present technology involves expressing the target protein (protein of interest) with an N-terminal fusion as depicted in FIG. 1(A). Briefly, the N-terminal tag comprises the following elements; a His-tag to bind to the affinity matrix (yellow), a small binding protein X (green), followed by a linker and a protease site with a methionine instead of the preferred amino acids at the P1′ position (blue). This N-terminal fusion is linked to the target protein (brown). The red arrow indicates the position of cleavage between the protease site and the methionine. This methionine constitutes the first amino acid of the native target protein. It is noted that although the schematic in FIG. 1(A) shows a “WELQ” site recognized by SplB protease, sites corresponding to other proteases may also be used.

Additionally, the corresponding protease is prepared (FIG. 1(B)), which is fused to binding protein Y (which binds binding protein X mentioned above) and a His-tag.

It is further noted that within the scope of the invention it is also possible for the relative positions of these components to be changed without affecting the basic concept.

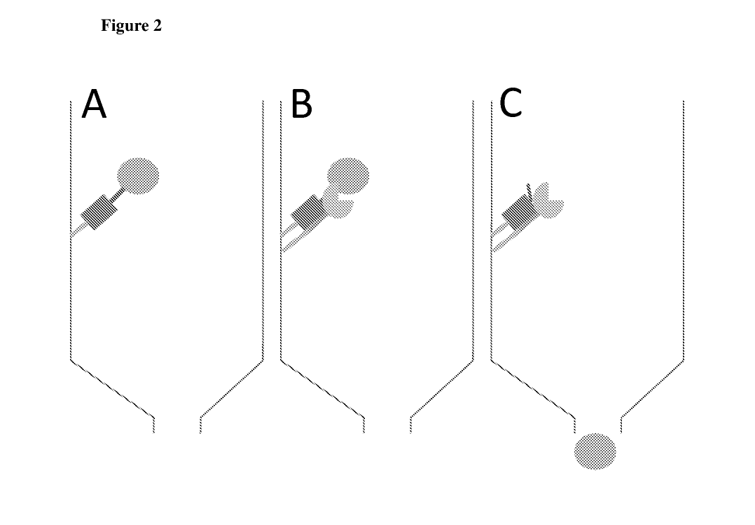

The N-terminal tag-target protein fusion is expressed by conventional means, the expressing cells are lysed and the lysate is contacted with an IMAC affinity column, where the expressed fusion protein binds while the non-specific proteins are washed away (FIG. 2(A)). Thereafter, the protease/binding protein Y/His-tag fusion protein is contacted with the target fusion protein bound to the affinity matrix. Binding proteins X and Y bind each other, thereby bringing the protease into close proximity of its sub-optimal site located N-terminal of the protein of interest (FIG. 2(B)). Due to the high local concentration of the protease enabled by the binding of proteins X and Y, the protease is nevertheless able to cleave its sub-optimal site and as a result of this cleavage the target protein will be released (FIG. 2(C)).

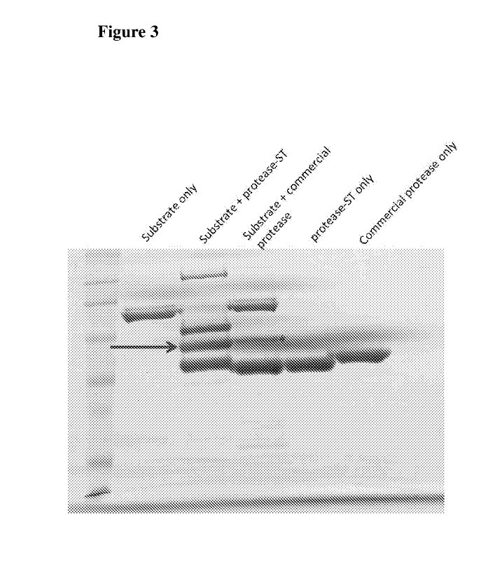

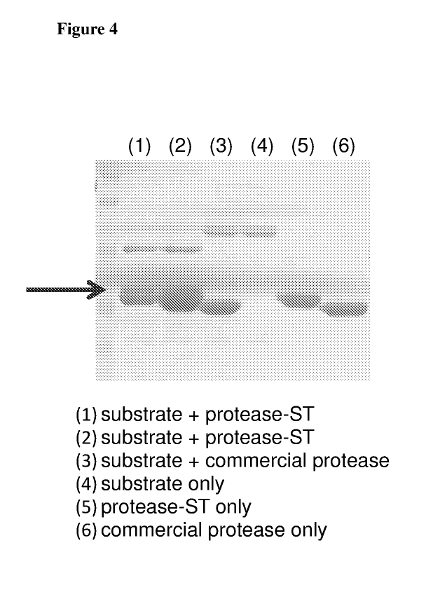

The above principle has been put into practice by mixing a purified protein comprising a protein named Spycatcher (binding protein X) followed by a SplB protease site with a P1′ methionine (WELQIM) and the lactamase Tem1. This protein was added to a SplB protease fused to Spytag (binding protein Y). As shown in FIG. 3, this led to the cleavage of Tem1 at the first methionine. Commercial SplB protease, lacking the fused spytag, was only able to produce minimal native Tem1. The precise cleavage site was confirmed by Mass Spectrometry. A similar experiment with LSSmOrange replacing Tem1 yielded the same result (FIG. 4).

### Example 2

### Target Protein Cleavage Using the Binding Pair of ARVCF or Truncated Versions Thereof and ePDZ-b

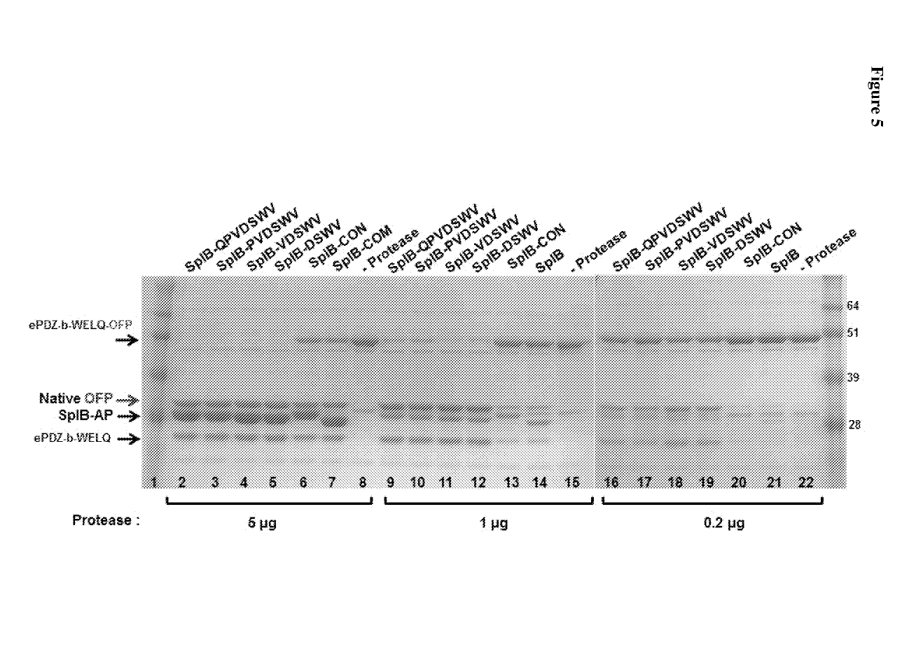

First, the principle was tested using ePDZ-b fused to the target protein (orange fluorescent protein, OFP) and ARVCF peptide fused to SplB protease (SplB-AP). SplB protease cleaves after the sequence WELQ with methionine at the P1′ position poorly tolerated. When combined with potential steric exclusion by the protein of interest being purified, methionine at P1′ will pose barriers to optimal SplB protease cleavage. The WELQ peptide sequence was introduced between ePDZ-b and OFP. Incubation of the fusion substrate (ePDZ-b-WELQ-OFP) with a stoichiometric excess of either SplB-AP or commercially available SplB protease (SplB-COM) resulted in cleavage and generation of native OFP (FIG. 5). However, this was notably more efficient for SplB-AP compared to SplB-COM and SplB fused to a control peptide that does not interact with ePDZ-b (SplB-CON) (cf. lanes 2, 6-8). Neither SplB-COM or Spl-CON was able to completely digest the fusion substrate. The increased efficiency of SplB-AP was more pronounced when it was reduced to sub-stoichiometric levels compared to substrate (FIG. 5, cf. lanes 9, 13-15 and lanes 16, 20-22). The very high affinity between ePDZ-b and ARFCP peptide may result in prolonged tethering of protease to ePDZ-b after cleavage of target protein. This would reduce “turn-over” of the protease, necessitating use of higher stoichiometric amounts. This hypothesis was tested by reducing the affinity of the ePDZ-b-ARVCF peptide interaction by serially truncating the ARVCF peptide fused to SplB from 8 to 4 amino acids (PQPVDSWV to DSWV). At high protease concentration, no variation was observed in cleavage efficiency (FIG. 5, lanes 2-5). At sub-stoichiometric amounts, 3 and 4 amino acid truncations of the ARVCF peptide showed clear improvements in activity compared to full-length peptide (FIG. 5, cf. lane 16 with 18-19). Furthermore, the overall activity compared to the SplBCON and SplB-COM was significantly enhanced (cf. lanes 18-19 and 20-21).

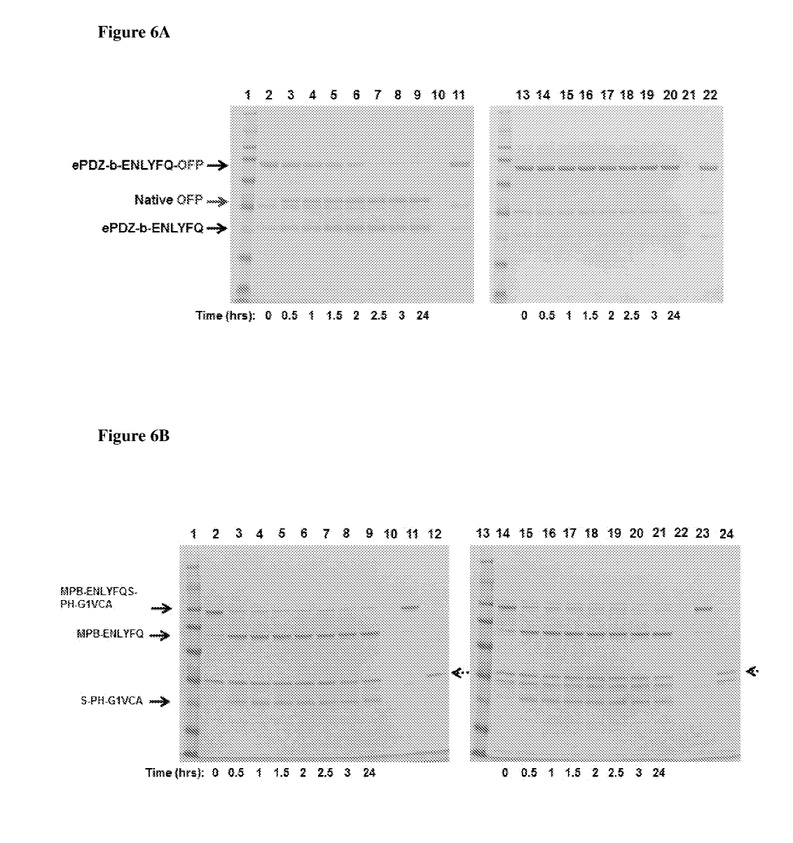

The same principle as described above was applied to TEV protease, one of the most ubiquitous enzymes used to remove affinity tags that optimally cleaves the consensus sequence ENLYFQIS. A fusion substrate was constructed wherein this sequence was truncated to ENLYFQ, and placed between the ePDZ-b and OFP components. Here, both steric constraints and a sub-optimal cleavage site (ENLYFQIM) would be expected to impact negatively on cleavage by wild-type TEV protease. The results show clearly improved cleavage when TEV is fused to the optimised 4-amino acid truncated ARVCF peptide (TEV-AP4) (FIG. 6A). Notable cleavage was observed after only 30 minutes incubation with near completion around 2.5 hours. In comparison, wild-type TEV protease did not show significant cleavage even after 24 hours incubation. A control experiment using a fusion substrate comprising the full TEV consensus sequence (ENLYFQS) led to equivalent cleavage by both wild-type TEV and TEV-AP4 (FIG. 6B).

### Example 3

### Applying the Concept of the Present Invention to On-Column Cleavage

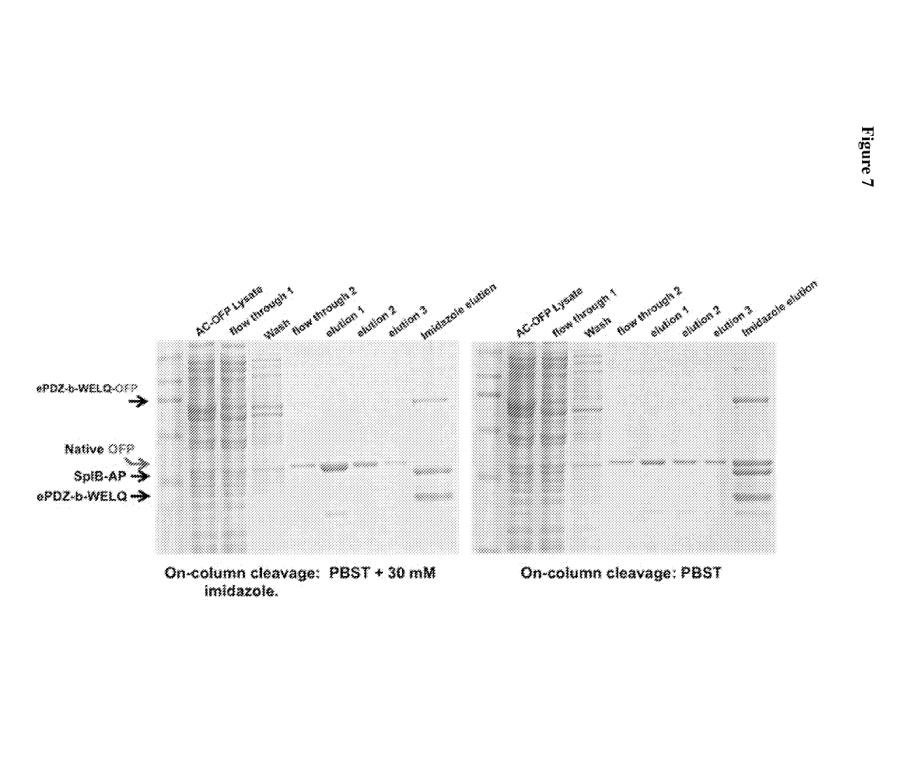

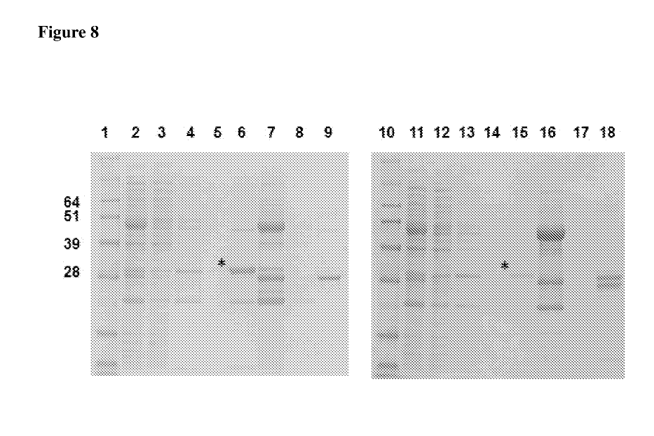

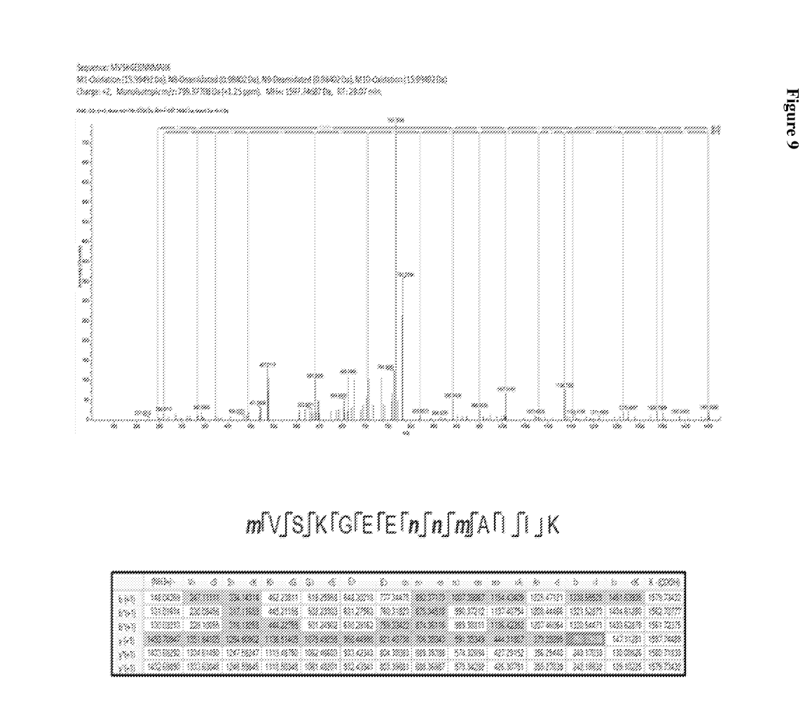

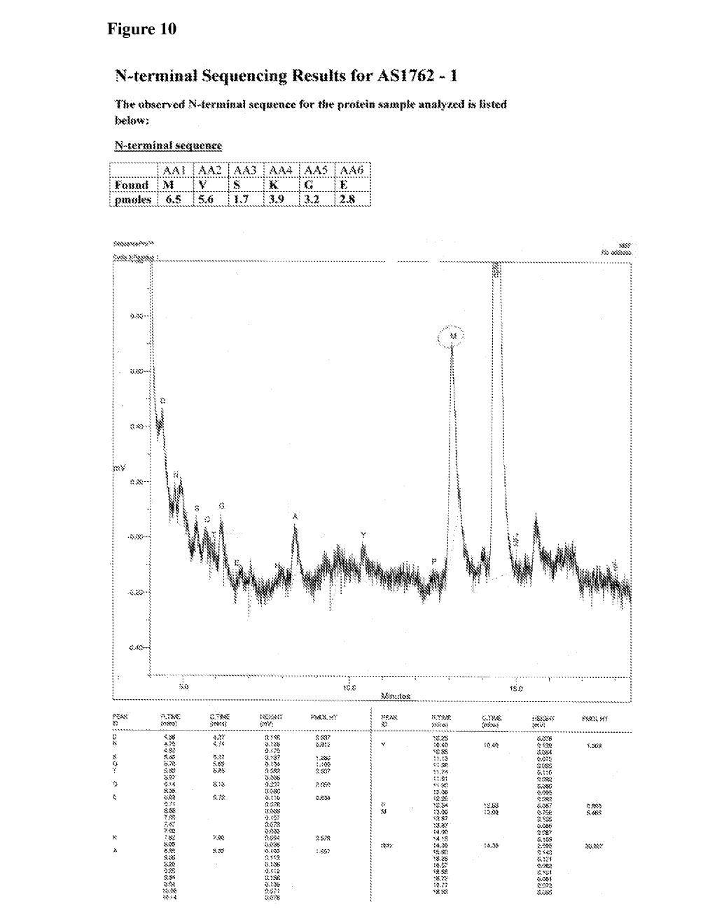

It was explored whether the enforced-proximity concept was applicable to conventional on-column cleavage and purification protocols using histidine tagged proteins. Complete immobilisation of protease via its histidine tag could reduce turnover during on-column cleavage of a co-immobilised substrate, necessitating use of increased amounts for efficient cleavage. Addition of 30 mM imidazole alleviated this constraint, resulting in improved cleavage efficiencies using histidine tagged ePDZ-b -WELQ-OFP and SplB-AP proteins (FIG. 7). These conditions were used for the on-column cleavage of histidine tagged ePDZ-b -ENLYFQ-OFP protein by histidine tagged TEV-AP4. Upon elution with PBS, the yield of native OFP was significantly increased when histidine tagged TEV-AP4 was used compared to histidine tagged TEV (FIG. 8, compare lanes 6 and 15). Both mass spectrophotometry and N-terminal sequencing analysis confirmed correct cleavage by TEV-AP4 to yield OFP with an N-terminal methionine (FIGS. 9 and 10).

The invention has been described broadly and generically herein. Each of the narrower species and subgeneric groupings falling within the generic disclosure also form part of the invention. This includes the generic description of the invention with a proviso or negative limitation removing any subject-matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1. Isolated polypeptide comprising
(a) a protein of interest;
(b) a first member of a pair of binding partners;
(c) an affinity tag for immobilizing the polypeptide on a solid support; and
(d) a modified endoprotease recognition site,
wherein the modified endoprotease site is located directly adjacent to the N-terminal amino acid of the protein of interest and comprises or only consists of the amino acid sequence N-terminal of the cleavage site of the native endoprotease recognition site.

2. The isolated polypeptide according to claim 1, wherein the first member of the pair of binding partners is located N-terminal to the modified protease recognition site.

3. The isolated polypeptide according to claim 1 or 2, wherein the affinity tag is located on the N- or C-terminus of the polypeptide, preferably the N-terminus.

4. The isolated polypeptide according to any one of claims 1-3, wherein the polypeptide has in N- to C-terminal orientation the general formula (I)
A-X-C-POI (I),
wherein
A represents the affinity tag;
X represents the first member of the pair of binding partners;
C represents the modified protease recognition site;
POI represents the protein of interest; and
“-” represents a peptide linker or peptide bond, wherein C and POI are linked by a peptide bond.

5. The isolated polypeptide according to any one of claims 1-4, wherein the affinity tag is selected from the group consisting of a 6× His-tag, glutathione-S-transferase (GST) tag, chitin binding domain (CBD), calmodulin binding peptide (CBP), and maltose binding protein (MBP).

6. The isolated polypeptide according to any one of claims 1-5, wherein the first member of the pair of binding partners is a peptide or polypeptide.

7. The isolated polypeptide according to claim 6, wherein the pair of binding partners is a pair of binding proteins or peptides.

8. The isolated polypeptide according to any one of claims 1-7, wherein the first member of a pair of binding partners is any member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrom (ARVCF) peptides (SEQ ID Nos. 7-9) as well as C-terminal fragments of the ARVCF peptides, and (iv) coiled coil (poly)peptide pairs.

9. The isolated polypeptide according to any one of claims 1-8, wherein the modified endoprotease recognition site is derived from staphylococcal serine protease-like B (SplB) protease, human rhinovirus 3C (HRV3C) protease, tobacco etch virus (TEV) protease and tobacco vein mottling virus (TVMV) protease recognition sites.

10. The isolated polypeptide according to claim 9, wherein the modified endoprotease recognition site is derived from
(1) an SplB protease recognition site and has the amino acid sequence WELQ (SEQ ID NO:1) or a derivative thereof; or
(2) an HRV3C protease recognition site and has the amino acid sequence LEVLFQ (SEQ ID NO:2) or a derivative thereof; or
(3) a TEV protease recognition site and has the amino acid sequence ENLYFQ (SEQ ID NO:3) or a derivative thereof; or
(4) a TVMV protease recognition site and has the amino acid sequence ETVRFQ (SEQ ID NO:4) or a derivative thereof.

11. The isolated polypeptide according to claim 10, wherein the derivatives of the modified endoprotease recognition sites comprise 1 or 2 amino acid substitutions relative to the amino acid sequences set forth in SEQ ID Nos. 1-4.

12. The isolated polypeptide according to any one of claims 1-11, wherein the N-terminal amino acid of the protein of interest is a methionine (M) residue.

13. Nucleic acid molecule encoding the polypeptide according to any one of claims 1-12.

14. The nucleic acid molecule according to claim 13, wherein the nucleic acid molecule is comprised in a vector, preferably an expression vector.

15. Host cell comprising the nucleic acid molecule of claim 13 or 14.

16. Method for isolating a protein of interest, comprising
(a) expressing the protein of interest in form of a fusion protein according to any one of claims 1-12 in a suitable expression system;
(b) contacting the fusion protein obtained in step (a) with a protease fusion protein, wherein the protease fusion protein comprises a protease domain capable of recognizing and cleaving the modified protease recognition site and the second member of the pair of binding partners, under conditions that allow binding of the fusion protein and the protease fusion protein by binding of the pair of binding partners and cleavage of the modified protease recognition site, thereby releasing the protein of interest from the fusion protein; and
(c) isolating the protein of interest.

17. The method according to claim 16, wherein the protease fusion protein further comprises an affinity tag identical to that of the fusion protein comprising the protein of interest.

18. The method according to claim 16 or 17, wherein the fusion protein is expressed in a cellular expression system.

19. The method according to claim 18, wherein the fusion protein is expressed by cultivating the host cell according to claim 15 under conditions that allow expression of the fusion protein.

20. The method according to any one of claims 16-19, wherein prior to step (b) the expressed fusion protein is at least partially purified.

21. The method according to claim 20, wherein the at least partial purification is carried out by subjecting the expressed fusion protein to affinity chromatography under conditions that allow immobilization of the fusion protein by interaction of the affinity tag with the solid affinity chromatography matrix.

22. The method according to claim 21, wherein step (b) is carried out while the fusion protein is immobilized on an affinity chromatography material.

23. The method according to any one of claims 16-22, wherein step (c) comprises separating the cleaved protein of interest from the remainder of the fusion protein, preferably by eluting the released protein of interest from an affinity chromatography matrix on which the fusion protein has been immobilized.

24. The method according to any one of claims 16-23, wherein the protease is SplB protease, HRV3C protease, TEV protease or TVMV protease.

25. The method according to any one of claims 16-24, wherein the second member of the pair of binding partners is a peptide or polypeptide.

26. The method according to claims 25, wherein the pair of binding partners is a pair of binding proteins or peptides.

27. The method according to any one of claims 16-26, wherein the second member of a pair of binding partners is the other member of the pairs of binding partners selected from the group consisting of (i) a binding pair of a small peptide, a small molecule or a DNA aptamer and a polypeptide target; (ii) a split domain of the FbaB-type fibronectin-binding protein of Streptococcus pyogenes (SEQ ID Nos. 5 and 6) or a functional fragment or derivative thereof, (iii) affinity clamp proteins and armadillo repeat gene deleted in velo-cardio-facial syndrom (ARVCF) peptide (SEQ ID Nos. 7-11) as well as C-terminal fragments of the ARVCF peptides, and (iv) coiled coil (poly)peptide pairs.

28. The method according to any one of claims 16-27, wherein the protease specifically recognizes and cleaves the modified protease recognition site.

29. The method according to any one of claims 16-28, wherein the fusion protein comprising the protein of interest or the protein of interest do not comprise another site recognized and cleaved by the protease.

30. Kit for protein purification, comprising
(a) an expression vector comprising a nucleic acid sequence encoding for an affinity tag, one member of a pair of binding partners and a modified endoprotease recognition site that allows generating a nucleic acid molecule according to claim 14 by cloning a nucleic acid sequence encoding for a protein of interest into said expression vector; and
(b) a protease fusion protein comprising a protease domain capable of recognizing and cleaving the modified protease recognition site and the other member of the pair of binding partners and optionally an affinity tag identical to that encoded by the expression vector.

31. Isolated polypeptide comprising
(a) a protein of interest, and
(b) an amino acid sequence as set forth in SEQ ID NO:10 or SEQ ID NO:11.

32. The isolated polypeptide of claim 31, wherein the protein of interest is a protease.

33. Method for degrading a target protein, comprising
providing a fusion protease protein, wherein the fusion protease protein comprises (a) a protease and (b) a target protein binding element,
contacting the fusion protease protein with the target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a),
wherein the target protein is degraded upon enforced interaction of the fusion protease protein and the target protein.

34. The method of claim 33, wherein the target protein binding element is selected from the group consisting of a peptide, an antibody or a fragment thereof, an aptamer and a small molecule.

35. Method for treatment of a disease,
wherein a pathogenic target protein is degraded by a fusion protease protein, the method comprising
providing the fusion protease protein, wherein the fusion protease protein comprises (a) a protease and (b) a target protein binding element,
contacting the fusion protease protein with the pathogenic target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a),
wherein the target protein is degraded upon enforced interaction of the fusion protease protein and the target protein.

36. Fusion protease protein for use as a medicament,
wherein a pathogenic target protein is degraded by a fusion protease protein, the method comprising
providing the fusion protease protein, wherein the fusion protease protein comprises (a) a protease and (b) a target protein binding element,
contacting the fusion protease protein with the pathogenic target protein, wherein the target protein comprises at least one amino acid sequence that has 40% -90% sequence homology over the whole length to a recognition site of the protease of (a) and does not contain a sequence that has 90% -100% sequence homology over the whole length to a recognition site of the protease of (a),
wherein the target protein is degraded upon enforced interaction of the fusion protease protein and the target protein.
